(19) United States
(12) Patent Application Publication
(10) Pub. No.: US 2012/0282337 A1
(43) Pub. Date: Nov. 8, 2012

(54) Modified Release Formulation

(75) Inventors: Thomas FRIEDL, Ochsenhausen (DE); Wolfram EISENREICH, Ulm (DE)

(73) Assignee: BOEHRINGER INGELHEIM INTERNATIONAL GMBH, Ingelheim (DE)

(21) Appl. No.: 13/547,343

(22) Filed: Jul. 12, 2012

Related U.S. Application Data
(63) Continuation of application No. 13/036,928, filed on Feb. 28, 2011, now abandoned.
Application No. 13/036,928 is a continuation of application No. 12/278,846, filed on Sep. 12, 2008, now abandoned.

(30) Foreign Application Priority Data
Feb. 10, 2006 (EP) 06002775.2

(51) Int. Cl. A61K 31/428; C07D 277/82; A61P 25/00; A61P 25/18; A61P 25/16; A61P 25/24; A61K 9/22; A61P 25/20

(52) U.S. Cl. 424/468; 514/367; 548/164

(57) ABSTRACT

The invention is directed to the use of an extended release tablet formulation for pramipexole.

### FIELD OF THE INVENTION

The invention is directed to the use of an extended release tablet formulation for pramipexole.

### BACKGROUND OF THE INVENTION

Pramipexole is a known dopamine D2 receptor agonist. It is structurally different from the ergot-derived drugs, e.g. bromocriptine or pergolide. It is also pharmacologically unique in that it is a full agonist and has receptor selectivity for the dopamine D2 family of dopamine receptors.

Pramipexole is designated chemically as (S)-2-Amino-4,5,6,7-tetrahydro-6-(propylamino)benzothiazole and has the molecular formula C₁₀H₁₇N₃S and a relative molecular mass of 211.33. The chemical formula is as follows:

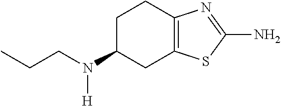

The salt form commonly used is pramipexole dihydrochloride monohydrate (molecular formula C₁₀H₂₁Cl₂N₃OS; relative molecular mass 302.27). Pramipexole dihydrochloride monohydrate is a white to off-white, tasteless, crystalline powder. Melting occurs in the range of 296° C. to 301° C., with decomposition. Pramipexole is a chiral compound with one chiral centre. Pure (S)-enantiomer is obtained from the synthetic process by chiral recrystallization of one of the intermediates during synthesis.

Pramipexole dihydrochloride monohydrate is a highly soluble compound. Water solubility is more than 20 mg/ml and solubility in buffer media is generally above 10 mg/ml between pH 2 and pH 7.4. Pramipexole dihydrochloride monohydrate is not hygroscopic, and of highly crystalline nature. Under milling the crystal modification (monohydrate) does not change. Pramipexole is very stable in the solid state, yet in solution it is light sensitive.

Pramipexole immediate release (IR) tablets were first authorised in the USA in 1997, followed over the course of the next years by marketing authorisations in the European Union (EU), Switzerland, Canada and South America as well as in countries in Eastern Europe, Near East and Asia.

Pramipexole IR tablets are indicated in the EU and US for the treatment of signs and symptoms of either early parkinson's disease or advanced parkinson's disease in combination with levodopa. A typical immediate release tablet (e.g. one known in Germany under the trade name Sifrol®) comprises as inactive ingredients mannitol, corn starch, colloidal silicon dioxide, povidone, and magnesium stearate and 0.125 mg, 0.25 mg, 0.5 mg or 1.0 mg, of pramipexole dihydrochloride monohydrate. Such a tablet is meant in the context whenever reference is made to an immediate release formulation of pramipexole and not otherwise defined. The IR tablets have to be taken 3 times a day.

From the pharmacokinetic point of view pramipexole IR tablets are rapidly and completely absorbed following oral administration. The absolute bioavailability is greater than 90% and the maximum plasma concentration occurs within 1 to 3 hours. The rate of absorption is reduced by food intake but not the overall extent of absorption. Pramipexole shows linear kinetics and a relatively small inter-patient variation of plasma levels. The elimination half-life (t_{1/2}[h]) varies from 8 hours in the young to 12 hours in the elderly.

As commonly known, modified release of active ingredient(s) allows to simplify the patient's administration scheme by reducing the amount of recommended daily intakes, improves patient's compliance, and attenuates adverse events, e.g. related to high plasma peaks. Modified release pharmaceutical preparations regulate the release of the incorporated active ingredient or ingredients over time and comprise formulations with a controlled, a prolonged, a sustained, a delayed, a slow or an extended release, so they accomplish therapeutic or convenience objectives not offered by conventional dosage forms such as solutions or promptly dissolving dosage forms.

A modified or extended release of active ingredient(s) from a pharmaceutical preparation may be accomplished by homogeneously embedding said active ingredient(s) in a hydrophilic matrix, being a soluble, partially soluble or insoluble network of viscous, hydrophilic polymers, held together by physical or chemical entanglements, by ionic or crystalline interactions, by complex formation, by hydrogen bonds or van der Waals forces. Said hydrophilic matrix swells upon contact with water, thereby creating a protective gellayer from which the active ingredient(s) is (are) slowly, gradually, continuously released in time either by diffusion through the polymeric network, by erosion of the gellayer, by dissolution of the polymer, or by a combination of said release mechanisms.

However, it may appears to be difficult to formulate a tablet having a suitable combination of modified, extended or sustained-release and handling properties, where the drug is one having relatively high solubility.

There are a number of approaches described in prior art to provide sustained release tablet compositions of pramipexole:

WO 2004/010997 describes a sustained-release pharmaceutical composition in a form of an orally deliverable tablet comprising a water-soluble salt of pramipexole, dispersed in a matrix comprising a hydrophilic polymer and a starch having a tensile strength of at least about 0.15 kN cm⁻², preferably at least about 0.175 kN cm⁻², and more preferably at least about 0.2 kN cm⁻², at a solid fraction representative of the tablet. The disclosure thereof is concentrated to provide a composition with sufficient hardness yield during a high-speed tableting operation, in particular to resist erosion during application of a coating layer. According to a preferred embodiment it is provided a pharmaceutical composition in a form of an orally deliverable tablet having a core comprising pramipexole dihydrochloride monohydrate in an amount of about 0.375, 0.75, 1.5, 3 or 4.5 mg, dispersed in a matrix comprising (a) HPMC type 2208 in an amount of about 35% to about 50% by weight of the tablet and (b) a pregelatinized starch having a tensile strength of at least about 0.15 kN cm⁻² at a solid fraction of 0.8, in an amount of about 45% to about 65% by weight of the tablet; said core being substantially enclosed in a coating that constitutes about 2% to about 7% of the weight of the tablet, said coating comprising an ethylcellulose-based hydrophobic or water-insoluble component and an HPMC-based pore-forming component in an amount of about 10% to about 40% by weight of the ethylcellulose-based component.

Furthermore, WO 2004/010999 discloses an orally deliverable pharmaceutical composition comprising a therapeutically effective amount of pramipexole or a pharmaceutically acceptable salt thereof and at least one pharmaceutically acceptable excipient, said composition exhibiting at least one of (a) an in vitro release profile wherein on average no more than about 20% of the pramipexole is dissolved within 2 hours after placement of the composition in a standard dissolution test; and (b) an in vivo pramipexole absorption profile following single dose oral administration to healthy adult humans wherein the time to reach a mean of 20% absorption is equal to or greater than about 2 hours and/or the time to reach a mean of 40% absorption is equal to or greater than about 4 hours. However, in practical use, it appears that any formulation having an extended or controlled release profile designed for a once daily application would meet the above requirements for which a general teaching how to adjust such a profile is missing.

It is an object of the present invention to provide a controlled release tablet composition of pramipexole or a pharmaceutically acceptable salt thereof that is suitable for once-daily oral administration. It is a further object to provide a tablet composition comprising pramipexole or a pharmaceutically acceptable salt thereof that provides a day-long therapeutic effect and will allow patients to treat their symptoms with a single daily dose, which makes it possible to adjust the release profile of the active ingredient according to a selected release profile dependent or independent from the pH values. Furthermore a method of manufacturing the tablet formulation shall be provided.

### DESCRIPTION OF THE INVENTION

Surprisingly, it has been found that pramipexole or a pharmaceutically acceptable salt thereof may be used in formulations as once daily extended (or slow) release tablets and two alternative formulation principles allow different release rate types dependent or independent from the pH value.

The present invention relates to an extended release tablet formulation comprising pramipexole or a pharmaceutically acceptable salt thereof in a matrix comprising at least one water swelling polymer other than pregelatinized starch.

Preferably the invention relates to an extended release tablet formulation, wherein the matrix comprises at least two water swelling polymers other than pregelatinized starch, and wherein at least one of the at least two polymers is an anionic polymer.

Also preferred is an extended release tablet formulation, wherein the anionic polymer is selected from the group of optionally crosslinked acrylic acid polymers, methacrylic acid polymers, alginates, and carboxymethylcellulose.

Also preferred is an extended release tablet formulation, wherein the anionic polymer is an optionally crosslinked acrylic acid polymer, and wherein the content of the optionally crosslinked acrylic acid polymer in the matrix is from about 0.25 wt.-% to about 25 wt.-%, and preferably from about 0.5 wt.-% to about 15 wt.-%, and preferably from about 1 wt.-% to about 10 wt.-%.

Also preferred is an extended release tablet formulation, wherein at least one of the at least two polymers is a substantially neutral polymer other than pregelatinized starch.

Also preferred is an extended release tablet formulation, wherein the substantially neutral polymer is selected from hydroxypropylcellulose and hydroxypropylmethylcellulose.

Particularly preferred is an extended release tablet formulation, wherein the substantially neutral polymer is hydroxypropyl methylcellulose, and wherein the content of hydroxypropyl methylcellulose in the matrix is from about 10 wt.-% to about 75 wt.-% and preferably from about 25 wt.-% to about 65 wt.-%.

Particularly preferred is an extended release tablet formulation, wherein the matrix comprises about:

| | | |
|---|---|---|
| (a) pramipexole or a salt thereof | 0.05 to 5 | wt.-% |
| (b) anionic water swelling polymer(s) | 0.25 to 25 | wt.-% |
| (c) neutral water swelling polymer(s) | 10 to 75 | wt.-% |
| (d) further excipients | ad 100 | wt.-% |

Particularly preferred is an extended release tablet formulation consisting of pramipexole-dihydrochloride monohydrate, Hypromellose 2208, Corn starch, Carbomer 941, Colloidal silicon dioxide and Magnesium stearate.

A preferred embodiment of the present invention relates to an extended release tablet formulation comprising pramipexole or a pharmaceutically acceptable salt thereof in a matrix comprising
(a) at least one water swelling polymer other than pregelatinized starch and optionally excipients, the resulting tablet providing a pH-independent in vitro release characteristic in the range from pH 1 to 7.5, or
(b) at least one water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.5.

Most preferably the present invention relates to a matrix of the extended release tablet formulation comprising at least one water swelling polymer other than pregelatinized starch, preferably a water swelling essentially neutral polymer, a water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.

The extended release formulations according to the present invention intended for oral administration allow to select and estimate which in vitro release characteristic and timing of a formulation is most suitable to achieve the desired in vivo plasma profiles preferably with a once daily application. Therefore, a formulation principle with several variants has been developed for a single unit matrix tablet, i.e. formulations having different release rate types are provided and a different pH dependency is available. These alternative formulations are beneficial to patients as the extended release drug delivery will allow patients to treat their symptoms with a single daily dose, thereby increasing patient convenience and compliance.

The term “in vitro release characteristic” as used hereinbefore or hereinafter is directed to a release characteristic as obtained in a kind of normally used liquid medium for in vitro experiments wherein the release of active ingredient from the extended release formulation can occur, i.e. for example in in vitro dissolution media, but also in body fluids or simulated body fluids, more in particular in the gastro-intestinal fluids.

In the context of the present invention the term “extended” release should be understood in contrast to an immediate release, the active ingredient is gradually, continuously liberated over time, sometimes slower or faster, dependent or independent from the pH value. In particular, the term indicates that the formulation does not release the full dose of the active ingredient immediately after oral dosing and that the formulation allows a reduction in dosage frequency, following the definition for extended release, interchangeable with slow release. A slow or extended release, used synonymously with prolonged action, sustained release, or modified release, dosage form is a dosage form that allows a reduction in dosing frequency or a significant increase in patient compliance or therapeutic performance as compared to that presented as a conventional dosage form (e.g. as a solution or an immediate drug-releasing, conventional solid dosage form).

A release characteristic which is pH-independent indicates that the release characteristic is virtually the same in different pH media.

According to the teaching of the present invention, extended release tablet formulations are provided with different in vitro release profiles.

The extended release tablets of the present invention are believed to apply a swelling and partly eroding polymer matrix. Based on the assumed mechanisms, the release profile may roughly follow a square root of time to exponential in vitro release characteristic. Depending on the particular embodiment formulation a) is widely, preferably substantially independent from the pH value in the range from pH 1 to 7.5, and formulation b) is faster in simulated gastric juice having a pH<4.5, preferably <4, but are widely, preferably substantially independent from the pH value in the range from 4.5 to 7.5. A faster release in simulated gastric juice versus slower release in the intestinal fluid can be advantageous in cases where a loading dose effect from the dosage form is desired, whereas a widely or substantially pH independent release profile can be advantageous to reduce the risk of dose dumping and food effects. “Substantially” in the context of defining the impact of pH to the release profile, e.g. “substantially independent” or “substantially impacting the pH release profile” and the like, preferably means that the difference in mean release profile at a pH of 4.5 and a pH of 6.8 is equal or less to 25%, preferably <20%, more preferably <15%; more preferably <10%, most preferably <5%. Percent (%) refers to the amount of pramipexole or the used pramipexole salt which is released of the declared amount of pramipexole or the used pramipexole salt, in the formulation prior to release.

According to the present invention under “formulation a)” is understood the tablet formulation wherein the matrix comprises the composition as above-defined under a) and under “formulation b)” is understood the tablet formulation wherein the matrix comprises the composition as above-defined under b).

The water swelling polymer of the present invention represents at least one hydrophilic water swelling polymer constituting the extended release matrix which slowly releases the pramipexole or its salt as active ingredient. The polymer swells upon contact with aqueous fluid following administration, resulting in a viscous, drug release regulating gellayer. The viscosity of the polymer preferably ranges from 150 to 100,000 mPa·s (apparent viscosity of a 2% aqueous solution at 20° C.).

Examples of such polymers are water swelling substantially neutral polymers or water swelling anionic polymers.

The term “water swelling substantially neutral polymers” of the present invention comprises alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcellulose esters; other natural, semi-synthetic, or synthetic di-, oligo- and polysaccharides such as galactomannans, tragacanth, agar, guar gum, and polyfructans; methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide, preferably cellulose ether derivatives such as hydroxypropyl methylcellulose and hydroxypropyl cellulose, most preferred hydroxypropyl methylcellulose.

The term “water swelling anionic polymer” of the present invention comprises acrylic acid polymerisate, methacrylic acid copolymers, alginates, carrageenans, acacia, xanthan gum, chitin derivates such as chitosan, carmellose sodium, carmellose calcium, preferably acrylic acid polymeris ate.

Different viscosity grades of hydroxypropyl cellulose and hydroxypropyl methylcellulose are commercially available. Hydroxypropyl methylcellulose (HPMC) preferably used in the present invention has a viscosity grade ranging from about 3,500 mPa·s to about 100,000 mPa·s, in particular ranging from about 4,000 mPa·s to about 20,000 mPa·s and most in particular a viscosity grade of about 6,500 mPa·s to about 15,000 mPa·s (apparent viscosity of a 2% aqueous solution at 20° C.), e.g. hypromellose 2208 or 2206 (DOW, Antwerp, Belgium). HPMC type 2208 contains 19-24% by weight methoxy and 4-12% by weight hydroxypropoxy substituents.

Hydroxypropyl cellulose having a viscosity higher than 1,500 mPa·s (apparent viscosity of a 1% aqueous solution at 20° C.) is preferred, in particular hydroxypropyl cellulose having a viscosity in the range from about 1500 to about 3000 mPa·s, preferably from 4000 to 6500 mPa·s (2% aqueous solutions), e.g. the Klucel series such as Klucel M (Hercules, Wilmington, USA).

Without wishing to be bound by theory, there are believed to exist three main mechanisms by which pramipexole or a salt thereof can be released from a hydrophilic matrix: dissolution, erosion and diffusion. Pramipexole or its salt will be released by the dissolution mechanism when it is homogeneously dispersed in a matrix network of a soluble polymer. The network will gradually dissolve in the gastrointestinal tract, thereby gradually releasing its load. The matrix polymer can also gradually be eroded from the matrix surface, likewise releasing pramipexole or its salt in time. When pramipexole is processed in a matrix made up of an insoluble polymer, it will be released by diffusion: the gastro-intestinal fluids penetrate the insoluble, sponge-like matrix and diffuse back out loaded with drug.

Therefore, the water swelling polymers constituting the matrix, particularly in a matrix according to formulation a), mainly provide for the controlled pharmacokinetic release profile of the preparation. Depending on the amount of water swelling polymer(s) processed in the preparation, the release profile can be tuned, i.e. larger amounts of swelling polymer lead to a more pronounced sustained release effect and vice versa. Preferably, the amount of water swelling polymer in the present formulation ranges from about 10% to about 80% by weight.

In addition, when using a combination of polymers, the ratio of said polymers also influences the release profile of the preparation. A combination of different polymers offers the possibility of combining different mechanisms by which pramipexole is released from the matrix. Such combination facilitates control of the pharmacokinetic release profile of the preparation at will. For example, when using one or more water swelling polymers, in particular hydroxypropyl cellulose and hydroxypropyl methylcellulose, the weight percentage of hydroxypropyl methylcellulose preferably ranges from 25% to about 62%; the weight percentage of hydroxypropyl cellulose preferably ranges between 0% and about 16%.

Release of pramipexole or a salt thereof from a matrix containing hydroxypropyl cellulose and hydroxypropyl methylcellulose occurs by a combined set of release mechanisms. Due to the higher solubility of hydroxypropyl methylcellulose compared with hydroxypropyl cellulose, the former will gradually dissolve and erode from the matrix, whereas the latter will more act as a sponge-like matrix former releasing the active ingredient mainly by diffusion.

The extended release tablet formulation according to formulation a) is pH-independent. Therefore, the disadvantage that food related dose-dumping may be encountered is avoided. The problem of food related dose-dumping in fed patients can be attributed to a lot of factors such as the mechanical forces that are exerted by the stomach on its content and thus on an ingested preparation as well as the different pH regions of the gastro-intestinal tract. Since the pH values encountered in the gastro-intestinal tract vary not only with the region of the tract, but also with the intake of food, an extended release formulation preferably also has to provide an extended release profile and in particular has to avoid dose-dumping regardless whether the patient is in fasted or fed conditions.

According to the present invention the oral extended release formulation a) retains its pharmacokinetic release profile along its way through the gastro-intestinal tract so as to avoid undesirable fluctuations in drug plasma concentrations or complete dose-dumping, in particular avoids dose-dumping in different regions of the gastro-intestinal tract.

Beside pramipexole or a salt thereof, and the water swelling polymer(s), the formulation of the present invention may also optionally comprise further excipients, i.e. pharmaceutically acceptable formulating agents, in order to promote the manufacture, compressibility, appearance and taste of the preparation. These formulating agents comprise, for example, diluents or fillers, glidants, binding agents, granulating agents, anti-caking agents, lubricants, flavors, dyes and preservatives. Other conventional excipients known in the art can also be included.

The filler may be selected from soluble fillers, for example, sucrose, lactose, in particular lactose monohydrate, trehalose, maltose, mannitol and sorbitol. Different grades of lactose can be used. One type of lactose preferably used in the present invention is lactose monohydrate 200 mesh. Other lactose monohydrates, e.g. lactose monohydrate of the type DCL 11 can also be used. The notation DCL refers to “Direct Compression Lactose”. In case of a water soluble active ingredient, like the one described in this invention, more preferably water insoluble fillers, such as starch and starch derivates other than pregelatinized starch, e.g. corn starch, potato starch, rice starch or wheat starch, microcrystalline cellulose, dibasic calcium phosphate dihydrate and anhydrous dibasic calcium phosphate, preferably corn starch, can be used in addition or instead of the water soluble fillers. The total weight percentage of filler ranges between about 5% and about 75% by weight.

A glidant can be used to improve powder flow properties prior to and during tableting and to reduce caking. Suitable glidants include colloidal silicon dioxide, magnesium trisilicate, powdered cellulose, talc, tribasic calcium phosphate and the like. Colloidal silicon dioxide is preferably included as a glidant in an amount up to about 2%, preferably about 0.2% to about 0.8%, by weight of the tablet.

A lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, talc, hydrogenated vegetable oil, zinc stearate and the like. In one embodiment, magnesium stearate is included as a lubricant in an amount of about 0.1% to about 1.5%, preferably about 0.3% to about 1%, by weight of the tablet.

Among the optional formulating agents that further may be comprised in the matrix formulation there may be mentioned agents such as polyvidone; copovidone; starch; acacia; gelatin; seaweed derivatives, e.g. alginic acid, sodium and calcium alginate; cellulose, preferably microcrystalline cellulose, cellulose derivatives, e.g. ethylcellulose, hydroxypropylcellulose, hydroxypropylmethylcellulose, having useful dry or wet binding and granulating properties; and antiadherents such as talc and magnesium stearate.

According to a preferred embodiment of the present invention the matrix of the extended release tablet formulation of alternative a) comprises or essentially consists of hydroxypropyl methylcellulose, such as hypromellose, and further excipients. The amount of hydroxypropyl methylcellulose is preferably in the range from 10 to 75%, particularly preferred from 25 to 65% most preferred from 35 to 55% by weight. The amount of further excipients is preferably in the range from 90 to 25%, particularly preferred from 75 to 35%, most preferred from 65 to 45% by weight.

The expression “consisting essentially” is understood in the sense that it does not in principle exclude the presence, in addition to the mandatory components mentioned, of other components, the presence of which does not affect the essential nature of the formulation.

In some embodiments of the present invention it is provided a pH-dependent release profile, the release of pramipexole or its salt from the tablet and subsequent the absorption into the blood stream can vary during the passage of the dosage form along the gastro-intestinal tract. Thus, formulation b) provides a pH-dependent release characteristic wherein the release characteristic in the range of pH<4.5 is faster and a slower and further on pH-independent release characteristic in the range from 4.5≦pH≦7.5.

The above details for the water swelling polymer and selection and type of optional excipients may apply to formulation b), too.

Moreover, an anionic water swelling polymer, preferably an acrylic acid polymerisate is mandatorily present in formulation b), which is preferably selected from carbomer or carbopol® series, known acrylic acid polymerisates having high molecular weights. Particularly preferred are for example carbomer 941 (carbopol® 71 G, carbopol® 971) and carbomer 934 (carbopol® 974). The acrylic acid polymerisate is preferably present in the range of 0.25 to 25% by weight, particularly preferred 0.5 to 15% by weight, most preferred 1 to 10% by weight. The pH dependency of formulation b) results from the presence of an anionic water swelling polymer, particularly preferred from the presence of acrylic acid polymerisate which intends to swell in a greater extent in the acid pH range above pH 4.5 and in the alkaline pH range.

An increasing amount of acrylic acid leads to a decrease of the release rate. Therefore, adjusting the amount of acrylic acid polymerisate makes it possible to further tune the dissolution profiles as desired. To adjust the amount of acrylic acid polymerisate in the preferred range from 0.25 to 25% by weight provides the further advantage that the desired, resp. matching, dissolution profiles can be adjusted, resp. maintained, for a variety of formulations composed of different amounts and/or types of gel-forming agents, water swelling polymers, fillers, and dry binders.

According to a preferred embodiment of the present invention the matrix of the extended release tablet formulation comprises or essentially consists of hydroxypropyl methylcellulose, acrylic acid polymerisate and further excipients. The amount of hydroxypropyl methylcellulose is preferably in the range from 10 to 75%, particularly preferred from 25 to 65%, most preferred from 35 to 55% by weight. The amount of acrylic acid polymerisate is preferably as above-mentioned. The amount of additional excipients is preferably in the range from 90 to 25% particularly preferred from 75 to 35%, most preferred from 65 to 45% by weight. Optionally carboxymethylcellulose sodium may additionally be present preferably in the range from 5 to 50%, particularly preferred from 10 to 40%, most preferred from 15 to 30% by weight.

As active ingredient, pramipexole or a pharmaceutically acceptable salt thereof, may be present in any amount suitable for the desired treatment of a patient. A preferred salt of pramipexole is the dihydrochloride salt, most preferably in the form of the monohydrate. Usual amounts are from about 0.1 to about 5 mg pramipexole salt. According to a particularly preferred embodiment e.g. 0.750 mg pramipexole dihydrochloride monohydrate, corresponding to 0.524 mg anhydrous base, is used in the extended release tablet formulation according to the present invention. However, any other amount of active ingredient suitable for treatment may be used with the only proviso that the amount of pramipexole or salt is sufficient to provide a daily dose in one to a small plurality, for example one to about 4, of tablets to be administered at one time. Preferably the full daily dose is delivered in a single tablet. An amount of pramipexole salt, expressed as pramipexole dihydrochloride monohydrate equivalent, of about 0.1 to about 10 mg per tablet, or about 0.05% to about 5% by weight of the composition, will generally be suitable. Preferably an amount of about 0.2 to about 6 mg, more preferably an amount of about 0.3 to about 5 mg, per tablet is present. Specific dosage amounts per tablet e.g. include 0.375, 0.5, 0.75, 1.0, 1.5, 3.0 and 4.5 mg pramipexole dihydrochloride monohydrate. The amount that constitutes a therapeutically effective amount varies according to the condition being treated, the severity of said condition, and the patient being treated.

An extended release tablet formulation according to the present invention, has preferably the following composition:

| | | |
|---|---|---|
| pramipexole or a salt thereof | 0.05 to 5% | by weight |
| water swelling polymer(s) | 10 to 75% | by weight |
| acrylic acid polymerisate | 0 to 25% | by weight |
| optional further excipient(s) | ad 100% | by weight. |

Therefore, a particularly preferred extended release tablet formulation of the present invention consists of

0.1 to 2% by weight of pramipexole or a salt thereof;

25 to 65% by weight of hydroxypropyl methylcellulose;

0 to 40% by weight of carboxymethylcellulose sodium;

0 to 75% by weight of corn starch other than pregelatinized starch;

0 to 15% by weight of acrylic polymerisate, preferably carbomer 941;

0.5 to 50% by weight of excipients, preferably selected from the group consisting of colloidal silicon dioxide, magnesium stearate, lactose monohydrate, mannitol, microcrystalline cellulose, dibasic anhydrous calcium phosphate, hydroxyproylcellulose, povidone, copovidone, talc, macrogols, sodium dodecylsulfate, iron oxides and titanium dioxide.

According to the present invention starch other than pregelatinized starch, preferably corn starch if present, may impart several functions at the same time such as filler, glidant, and the like. However, it may be preferred to exclude starch completely from the tablet formulation according to the present invention, which may be replaced by one or more of the above-mentioned other excipient(s).

It is preferred that no coating is present on the tablet formulation according to the present invention. However, the extended release tablet of the invention may comprise a nonfunctional coating. A nonfunctional coating can comprise a polymer component, for example HPMC, optionally with other ingredients, for example one or more plasticizers, colorants, etc. The term “nonfunctional” in the present context means having no substantial effect on release properties of the tablet, and the coating serves another useful purpose. For example, such a coating can impart a distinctive appearance to the tablet, provide protection against attrition during packaging and transportation, improve ease of swallowing, and/or have other benefits. A nonfunctional coating should be applied in an amount sufficient to provide complete coverage of the tablet. Typically an amount of about 1% to about 10%, more typically an amount of about 2% to about 5%, by weight of the tablet as a whole, is suitable.

The tablets of the present invention can be of any suitable size and shape, for example round, oval, polygonal or pillow-shaped, and optionally bear nonfunctional surface markings. According to the present invention it is preferred that the extended release tablets are white to off-white and of oval or round, biconvex, shape.

Tablets of the invention can be packaged in a container, accompanied by a package insert providing pertinent information such as, for example, dosage and administration information, contraindications, precautions, drug interactions and adverse reactions.

The present invention is further directed to the use of the extended release tablet formulation according to the present invention for preparing a medical composition for the treatment of any of the following diseases: Bipolar Disorder, Fibromyalgia, Restless Legs Syndrom, Parkinson Disease, in particular idiopathic Parkinson Disease, more particular idiopathic Parkinson Disease in an advanced stage. Bipolar Disorder is a manic-depressive disease, in that manic-stages, depressive stages and mixed stages may occur. The disease is characterised of unusual shifts in a person's mood, energy, and ability to function.

Different from the normal ups and downs that everyone goes through, the symptoms of bipolar disorder are severe. They can result in damaged relationships, poor job or school performance, and even suicide. Scientifically one distinguishes between Bipolar I disorder, Bipolar II Disorder, Cyclothymia and Bipolar Disorders Not Otherwise Specified. In Bipolar I Disorder full-fledged manic and major depressive episodes alternate. Among the criteria for Bipolar I Disorder are: single manic episodes, most recent episode hypomanic, most recent episode manic, moist recent episode mixed, most recent episode depressed, most recent episode unspecified. Bipolar I disorder commonly begins with depression and is characterized by at least one manic or excited period during its course. The depressive phase can be an immediate prelude or aftermath of mania, or depression and mania can be separated by months or years.

Bipolar II Disorder are characterised by recurrent major depressive episodes with hypomanic episodes. Cyclothymida disorder is a chronic, fluctuating mood disturbance which involves periods of hypomanic symptoms, and periods of depressive symptoms.

In Bipolar II Disorder usually depressive episodes alternate with hypomanias (relatively mild, nonpsychotic periods of usually less than 1 week). During the hypomanic period, mood brightens, the need for sleep decreases, and psychomotor activity accelerates beyond the patient's usual level. Often, the switch is induced by circadian factors (eg, going to bed depressed and waking early in the morning in a hypomanic state). Hypersomnia and overeating are characteristic and may recur seasonally (eg, in autumn or winter); insomnia and poor appetite occur during the depressive phase. For some persons, hypomanic periods are adaptive because they are associated with high energy, confidence, and supernormal social functioning. Many patients who experience pleasant elevation of mood, usually at the end of a depression, do not report it unless specifically questioned. Skillful questioning may reveal morbid signs, such as excesses in spending, impulsive sexual escapades, and stimulant drug abuse. Such information is more likely to be provided by relatives.

Patients with major depressive episodes and a family history of bipolar disorders (unofficially called Bipolar III Disorder) often exhibit subtle hypomanic tendencies; their temperament is termed hyperthymic (ie, driven, ambitious, and achievement-oriented).

Fibromyalgia is an increasingly recognized chronic pain illness characterized by widespread musculoskeletal aches, pain and stiffness, soft tissue tenderness, general fatigue and sleep disturbances. The most common sites of pain include the neck, back, shoulders, pelvic girdle and hands, but any body part can be involved. Fibromyalgia patients experience a range of symptoms of varying intensities that wax and wane over time.

The disease is characterized by the presence of multiple tender points and a constellation of symptoms. Patients have widespread pain over all parts of the body which often seems to arise in the muscles. The pain is profound, widespread and chronic. The pain is described as deep muscular aching, throbbing, twitching, stabbing and shooting pain. Neurological complaints such as numbness, tingling and burning are often present and add to the discomfort of the patient. The severity of the pain and stiffness is often worse in the morning. Aggravating factors that affect pain include cold/humid weather, non-restorative sleep, physical and mental fatigue, excessive physical activity, physical inactivity, anxiety and stress. Additionally to pain, patients commonly complain of fatigue in form of an all-encompassing exhaustion that interferes with even the simplest daily activities. Within the spectrum of symptoms are a decreased sense of energy, disturbances of sleep, problems with memory and concentration and varying degrees of anxiety and depression. Furthermore, certain other medical conditions are commonly associated with fibromyalgia, such as: tension headaches, migraine, irritable bowel syndrome, overactive bladder, pelvic pain, premenstrual tension syndrome, cold intolerance, skin sensitivities and rashes, dry eyes and mouth, anxiety, depression, ringing in the ears, dizziness, vision problems, Raynaud's Syndrome, neurological symptoms, impaired coordination and restless leg syndrome. Patients with established rheumatoid arthritis, lupus (SLE) and Sjogren's syndrome often develop fibromyalgia during the course of their disease.

Restless Leg Syndrome, also known as RLS, anxietas tibiarum, Syndrom Wittmaack-Ekbom-Syndrom, often called paresthesias (abnormal sensations) or dysesthesias (unpleasant abnormal sensations), is a neurological disorder which manifests itself chiefly as sensory disorders of the legs such as tingling, dragging, tearing, itching, burning, cramp or pain and in those affected triggers an irresistible compulsion to move. These sensations usually occur deep inside the leg, between the knee and ankle; more rarely, they occur in the feet, thighs, arms, and hands. Although the sensations can occur on just one side of the body, they most often affect both sides.

Frequently these disorders occur when the affected person is resting. Particularly at night, during sleep, these sensory disorders and the consequent compulsive movements lead to restlessness and sleep disorders. As a result, most people with RLS have difficulty falling asleep and staying asleep. Left untreated, the condition causes exhaustion and daytime fatigue. Many people with RLS report that their job, personal relations, and activities of daily living are strongly affected as a result of their exhaustion. They are often unable to concentrate, have impaired memory, or fail to accomplish daily tasks.

The symptoms of RLS vary in severity and duration from person to person. Mild RLS occurs episodically, with only mild disruption of sleep onset, and causes little distress. In moderately severe cases, symptoms occur only once or twice a week but result in significant delay of sleep onset, with some disruption of daytime function. In severe cases of RLS, the symptoms occur more than twice a week and result in burdensome interruption of sleep and impairment of daytime function.

The disease may begin at any time in life. Elderly people are more often affected than the younger. Usually, the disease is a chronic disease,which starts in a mild form, but usually the symptoms amplify over time.

The disease may be associated with or patients may develop further conditions, f.e. patients also may suffer from periodic limb movement disorder (PLMD). PLMD is characterized by involuntary leg twitching or jerking movements during sleep that typically occur every 10 to 60 seconds, sometimes throughout the night. The symptoms cause repeated awakening and severely disrupted sleep. Unlike RLS, the movements caused by PLMD are involuntary, meaning the patient has no control over them. Although many patients with RLS also develop PLMD, most people with PLMD do not experience RLS. The invention refers also to RLS in children.

Advanced stage in idiopathic Parkinson's disease is accompanied by motor dysfunction as Parkinson's disease is considered to be a motor system disorder. The most frequent symptoms of PD are tremor, rigidity/akinesia, loss of dexterity, handwriting disturbances, gait disturbances, bradykinesia, postural instability, difficulty in swallowing and chewing, difficulties in speaking, urinary problems, constipation and / or other. Motor fluctuations may develop with the progression of the disease. Such changes are often referred to as late (motor)-complications of PD. Such late motor fluctuations and dyskinesia complications may have idiopathic origin as well as they may be caused by long-term dopaminergic treatment, f.e. with L-DOPA. In the progression of treatment with dopaminergic drugs side effects typically may increase over time, and the disease often manifests an “on-off” syndrome in advanced patients in which the drug simply doesn't work for unpredictable durations. In such stage periods with rapid fluctuations between uncontrolled movements and normal movement may occur, usually occurring after long-term use of L-DOPA. Advanced patients often have a “off”-time of more than 2 hours, more often more than 3 or even more than 4 hours a day.

The present invention is also interesting for to treat patients suffering from Parkinson's disease with dementia. In some instances of such patients, Magnetic Resonance Imaging (MIR), T1-weighted images or Computed Tomography (CT) Imaging reveal lesions in the cerebral white matter. They are not seen in parkinsonians without dementia.

A more systematic approach to define the stage of the Parkinson's disease are the modified Hoehn and Yahr scale or the Unified Parkinson Disease Rating Scale (UPDRS).

It may be considered that patients with a score of at least 2 to 3, preferably 3, more preferably 4 according the modified Hoehn and Yahr system are in an advanced stage of Parkinson's disease in the sense of the present invention. In this five stage disability scale stage one means least severe and stage five means most severe.

Stage One symptoms are signs and symptoms on one side only, symptoms mild, symptoms inconvenient but not disabling, usually presents with tremor of one limb, friends have noticed changes in posture, locomotion and facial expression.

Stage Two symptoms are symptoms are bilateral, minimal disability, posture and gait affected.

Stage Three symptoms are significant slowing of body movements, early impairment of equilibrium on walking or standing, generalized dysfunction that is moderately severe.

Stage Four symptoms are severe symptoms, can still walk to a limited extent, rigidity and bradykinesia, no longer able to live alone, tremor may be less than earlier stages.

Stage Five symptoms are cachectic stage, invalidism complete, cannot stand or walk, requires constant nursing care.

The Unified Parkinson Disease Rating Scale is a rating tool to follow the longitudinal course of Parkinson's Disease. It is made up of the following sections: 1) mentation, behavior, and mood, 2) activities of daily living and 3) motor. How to transfer this systematic to the severity of the disease can be taken from prior art. This system also may be used to define advanced stages of Parkinson's disease according to the present invention. In one embodiment, the formulation of the present invention can be used to treat patients with Parkinson's disease where depressed mood is the most cumbersome symptom. On the other hand the formulation is useful to treat motor symptoms of Parkinson's Disease.

It will be appreciated that it is up to the physician which kind of patients suffering from the disease he wants to treat with the active ingredient pramipexole, pramipexole dihydrochloride or another salt thereof respectively. According to the age of the elected patient, an adjustment of the dosage in the formulation of the invention will be necessary, in particular if children are to be treated.

The present invention is supposed to show less side effects than an immediate release formulation taken thrice daily, which provides about the same average pramipexole plasma concentration under comparable condition.

The term “comparable conditions” means that f.e. an oral immediate release dosage form is a Sifrol® tablet which has to be taken up to three times daily. If taken thrice daily in intervals which are constant over a period of 24 hours the average blood plasma concentration can be compared to an extended release formulation with a release characteristic over 24 hours.

Sifrol® is an oral administration tablet, which contains 0.125 mg, 0.25 mg, 0.5 mg or 1.0 mg of pramipexole dihydrochloride monohydrate, beside mannitol, corn starch, colloidal silicon dioxide, povidone, and magnesium stearate.

Accordingly, the extended release formulation is suited for the manufacture of a medication comprising pramipexole or a pharmaceutically acceptable salt thereof with a reduced side effect profile in terms of sleepiness and/or hallucinations and/or dizziness and/or headache and/or dyskinesia and/or obstipation and/or periphere oedema and/or nausea in comparison to an immediate release tablet, which is taken as often as needed to provide the same average blood plasma concentration over the release period of the extended release tablet taken once in the same period.

Furthermore, the present invention is preferably directed to a method of manufacturing the extended release tablet formulations via a direct compression process comprising the steps of

(1) producing an active ingredient trituration wherein the active ingredient is pramipexole or a pharmaceutically acceptable salt thereof by preblending it with a portion of water swelling polymer(s) and/or further excipient(s) in a mixer, wherein pramipexole or the pharmaceutically acceptable salt thereof is milled, preferably peg-milled, prior to use;

(2) premixing the active ingredient trituration of step (1), the main portion of the water swelling polymer(s) and/or excipients in a mixer to obtain a pre-mixture;

(3) optionally dry screening the pre-mixture through a screen in order to segregate cohesive particles and to improve content uniformity;

(4) mixing the pre-mixture of step (2) or (3) in a mixer, optionally by adding remaining excipients to the mixture and continuing mixing; and

(5) tableting the final mixture by compressing it on a suitable tablet press to produce matrix tablets.

Therefore, the tablets are manufactured via a direct compression process which applies to both types of pramipexole extended release matrix tablets. To achieve adequate content uniformity in this low drug load formulation, the active ingredient is preferably peg-milled. Preferably the particle size distribution of the peg-milled drug substance, as determined by laser diffractometry using a dry dispensing system, is characterized by particle fraction of 90% (V/V) being smaller than 100 μm, most preferably a particle fraction of 90% (V/V) being smaller than 75 μm in diameter.

Also other processes can be applied to the manufacturing of pramipexole extended release tablets, like conventional wet granulation and roller compaction. In case of wet granulation preferably pramipexole is granulated with suitable fillers, like e.g. starches other than pregelatinized starch, microcrystalline cellulose, lactose monohydrate or anhydrous dibasic calcium phosphate, and wet binding agents, like e.g. hydroxypropylmethyl cellulose, hydroxypropyl cellulose, povidone, copovidone, and starch paste, leading to a active ingredient concentrate, which after drying and dry screening is mixed with the main fraction of gel forming excipients, like all the above described retarding principles. In case of roller compaction, or in other words dry granulation, either a premix of pramipexole with part of the excipients used in the direct compression process, or the complete mixture containing all excipients, is processed through a conventional roller compactor to form ribbons, which are thereafter screened down to granules which are finally mixed with other excipients, like glidants, lubricants and antiadherents.

### BRIEF DESCRIPTION OF THE DRAWINGS

FIG. 1 is a flow diagram illustrating a preferred embodiment of the direct compression manufacturing process according to the present invention;

FIG. 2 is a graph illustrating the dissolution profiles of a matrix tablet formulation according to the present invention which contains 4% by weight carbopol® in 3 different pH media; and

FIG. 3 is a graph illustrating the dissolution profiles of 3 matrix tablet formulations according to the present invention which contain 0% , 1% and 4% by weight of carbopol®, respectively.

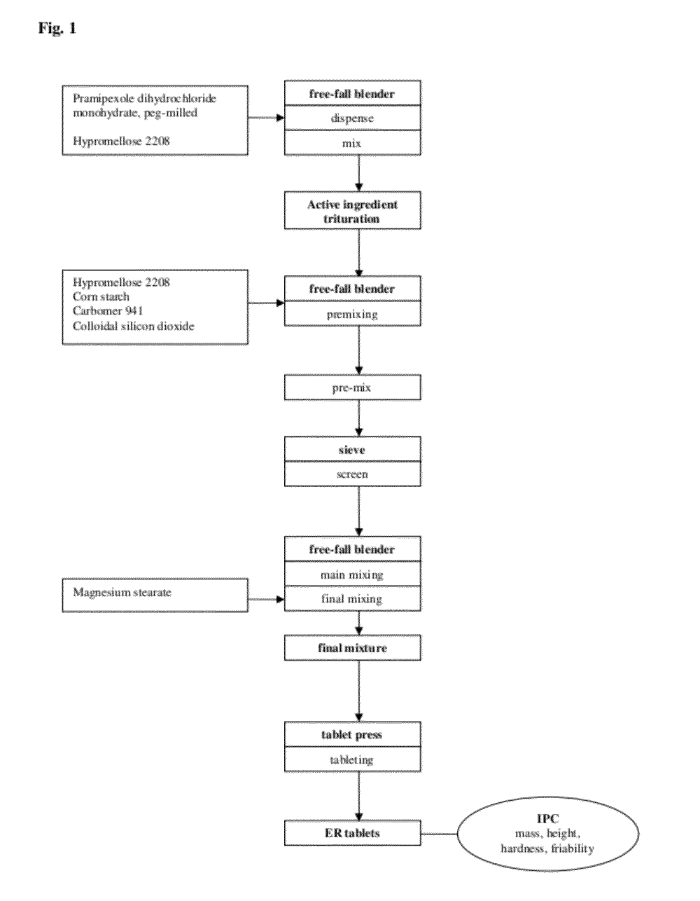

FIG. 1 illustrates a preferred embodiment of the manufacturing process with reference to a flow diagram wherein the manufacture of the extended release tablets of Examples 1 and 2 are exemplarily shown. FIG. 1 shows the detailed process steps and the in process controls performed. Process step 1 is optional. If omitted, the components of the formulation as described in process step 1 may be premixed with the remaining components of process step 2 without prior trituration.

Process step (1) is directed to the active ingredient trituration, i.e. in the present case a salt of pramipexole, pramipexole dihydrochloride monohydrate, in peg-milled quality, is preblended with a portion of the polymer, in this case hydroxypropyl methylcellulose, in a commonly known mixer. In the flow chart a Turbula free-fall mixer or blender is used. The mixing time is several minutes, in the present case preferably 10 min

In process step (2) according to the flow chart a premixing is performed, wherein the active ingredient trituration and the main portion of the water swelling polymer(s) and excipients are premixed for several minutes to obtain a pre-mix. In the present case the main portion of hydroxypropyl methylcellulose (hypromellose), corn starch, carbomer 941 and colloidal silicon dioxide are premixed for 5 min in the above-mentioned Turbula mixer or blender.

According to the following process step (3) a dry screening may optionally take place. The pre-mixture may be manually screened through a screen, for example a 0.8 mm mesh size screen, in order to segregate cohesive particles and to improve content uniformity.

In the subsequent process step (4) the main mixing step is performed according to which the components are mixed for several minutes, preferably 5 min. in the Turbula mixer after screening. Optionally further excipients may be added at this time, in the flow chart the component magnesium stearate is added to the main mixture, and further mixing for several minutes, e.g. 3 min., in the Turbula mixer is performed (final mixing) to obtain the final mixture.

Process step (5) of the process according to the present invention is the tableting. The final mixture is compressed on a suitable tablet press to produce, for example, oblong shaped matrix tablets (ER tablets=extended release tablets). In order to control and maintain the required quality the obtained matrix tablets are subjected to the following in-process controls: tablet mass, hardness, tablet height and friability.

The obtained pramipexole extended release tablets of the present invention may then be filled, for example, into High Density Polyethylene (HDPE) bottles. The bottles are closed tightly with screw caps and appropriately labelled, whereby all packaging and labelling activities are performed according to cGMP regulations. Alternatively, a blister type packaging can be used, e.g. using aluminium/aluminium foil blisters.

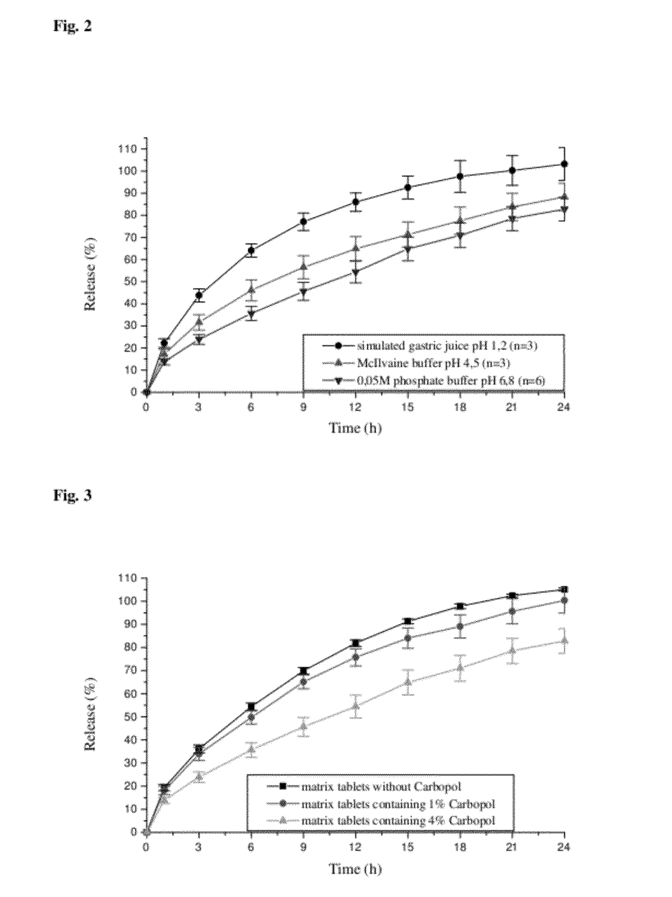

FIG. 2 represents a graph illustrating the dissolution profiles of a matrix tablet formulation according to the present invention. The matrix tablet contains 4% by weight carbopol®, the detailed composition is given in Example 2. The release characteristics of the matrix tablet in 3 different pH media are shown, i.e. in 0.05 M phosphate buffer, pH=6.8, n=x, in simulated gastric juice, pH=1.2, n=x, and in McIlvaine buffer, pH=4.5, n=x; (x represents the number of units tested). The value percent of released active ingredient is plotted against the time (hours).

FIG. 3 represents a graph illustrating the dissolution profiles of 3 matrix tablet formulations according to the present invention. The matrix tablets contain no carbopol®, 1% or 4% by weight carbopol®, respectively. The medium is a 0.05 M phosphate buffer, pH=6.8. The value percent of released active ingredient is plotted against the time (hours).

FIG. 2 shows a pH-dependent release characteristic wherein the release characteristic in the range of pH<4.5 is faster in case carbopol® is present. FIG. 3 shows, that an increase of the amount of carbopol® leads to a decreased releasing rate.

The advantages of the present invention are manifold:

According to the present invention, extended release tablets containing pramipexole or its salt are available showing different in vitro release profiles. It is possible to select a tailor-made release characteristic for patient's needs, symptoms and clinical picture observed.

The primary indication for pramipexole, Parkinson's disease, is an affliction that becomes more prevalent with advancing age and is often accompanied by decline in memory. Therefore, the matrix tablets according to the present invention providing an extended or slow release of pramipexole or a salt thereof allows to simplify the patient's administration scheme by reducing the amount of recommended daily intakes and improves patient's compliance, particularly relevant for elderly patients. The inventive extended release tablet formulation provides a daily dose preferably administered at one time.

Furthermore, the tablets of the present invention may be manufactured via a direct compression, wet or dry granulation process which applies to both types of extended release matrix tablets.

The invention described will now be illustrated by the Examples which follow various other embodiments and will become apparent to the skilled person from the present specification. However, it is expressly pointed out that the Examples and description are intended solely as an illustration and should not be regarded as restricting the invention.

### EXAMPLES

According to the present invention pramipexole extended release tablets have been manufactured. The tablets of the Examples are white to off-white, 14×6.8 mm oblong shaped, biconvex tablets. The tablets are intended to be administered orally, and shall not be divided into halves. The pramipexole tablets in the Examples contain 0.75 mg of pramipexole dihydrochloride monohydrate, corresponding to 0.524 mg of pramipexole free, anhydrous base.

### Example 1

TABLE 1

| Constituents | mg/tablet |
|---|---|
| Pramipexole-dihydrochloride monohydrate, peg-milled | 0.750 |
| Carbomer 941 (Carbopol ® 71 G) | 52.500 |
| Lactose monohydrate (200 mesh) | 140.000 |
| Calcium phosphate, dibasic dihydrate | 153.600 |
| Colloidal silicon dioxide | 1.400 |
| Magnesium stearate | 1.750 |
| Total weight matrix tablet | 350.000 |

### Example 2

TABLE 2

| Constituents | mg/tablet |
|---|---|
| Pramipexole-dihydrochloride monohydrate, peg-milled | 0.750 |
| Hypromellose 2208 (Methocel K 15 M) | 157.500 |
| Corn starch | 163.400 |
| Carbomer 941 (Carbopol ® 71 G) | 24.500 |
| Colloidal silicon dioxide | 2.100 |
| Magnesium stearate | 1.750 |
| Total weight matrix tablet | 350.000 |

### Example 3

TABLE 3

| Constituents | mg/tablet |
|---|---|
| Pramipexole-dihydrochloride monohydrate, peg-milled | 0.750 |
| Hypromellose 2910 (Methocel E 5) | 0.788 |
| Corn starch | 173.812 |
| Hypromellose 2208 (Methocel K 15 M) | 157.500 |
| Carbomer 941 (Carbopol ® 71 G) | 14.000 |
| Colloidal silicon dioxide | 1.400 |
| Magnesium stearate | 1.750 |
| Total weight matrix tablet | 350.000 |

### Example 4

TABLE 4

| Constituents | mg/tablet |
|---|---|
| Pramipexole-dihydrochloride monohydrate, peg-milled | 0.750 |
| Hypromellose 2208 (Methocel K 15 M) | 148.500 |
| Corn starch | 160.620 |
| Carbomer 941 (Carbopol ® 71 G) | 16.500 |
| Colloidal silicon dioxide | 1.980 |
| Magnesium stearate | 1.650 |
| Total weight matrix tablet | 330.000 |

### Example 5

One embodiment of the qualitative and quantitative composition of pramipexole extended release tablets according to the present invention is shown in TABLE 1.

TABLE 5
Qualitative and quantitative composition
of pramipexole extended release tablet

| | mg per | | |
|---|---|---|---|
| | 0.75 mg | | Reference to |
| Ingredient | tablet | Function | Standards |
| Pramipexole-dihydrochloride | 0.750 | Active | Corporate |
| monohydrate, peg-milled | | ingredient | standard |
| Hypromellose 2208 | 157.500 | Swelling | Ph. Eur./USP |
| (Methocel K 15 M) | | agent | |
| Corn starch | 183.700 | Filler | Ph. Eur./NF |
| Carbomer 941 | 3.500 | Gelling | Ph. Eur./NF |
| (Carbopol ® 71 G) | | agent | |
| Colloidal Silicon dioxide | 2.800 | Glidant | Ph. Eur./NF |
| Magnesium stearate | 1.750 | Lubricant | Ph. Eur./NF |

| | |
|---|---|
| Total | 350.000 |

### Example 6

A further embodiment of the qualitative and quantitative composition of pramipexole extended release tablets according to the present invention is shown in TABLE 2.

TABLE 6
Qualitative and quantitative composition
of pramipexole extended release tablet

| | mg per | | |
|---|---|---|---|
| | 0.75 mg | | Reference to |
| Ingredient | tablet | Function | Standards |
| Pramipexole-dihydrochloride | 0.750 | Active | Corporate |
| monohydrate, peg-milled | | ingredient | standard |
| Hypromellose 2208 | 157.500 | Swelling | Ph. Eur./USP |
| (Methocel K 15 M) | | agent | |
| Corn starch | 174.600 | Filler | Ph. Eur./NF |
| Carbomer 941 | 14.000 | Gelling | Ph. Eur./NF |
| (Carbopol ® 71 G) | | agent | |
| Colloidal Silicon dioxide | 1.400 | Glidant | Ph. Eur./NF |
| Magnesium stearate | 1.75 [symbol] | Lubricant | Ph. Eur./NF |
| Total | 350.000 | | |

[symbol]

indicates data missing or illegible when filed

### Example 7

The batch formula for the two pramipexole tablet formulations of Example 1 and 2 is shown in Table 3. The batch size of the final mixture corresponds to a batch size of 2000 tablets.

TABLE 7
Composition per batch of pramipexole 0.75 mg ER tablets

| | Grams per batch | Grams per batch |
|---|---|---|
| Ingredient | Example 1 | Example 2 |
| Pramipexole-dihydrochloride | 1.500 | 1.500 |
| monohydrate, peg-milled | | |
| Hypromellose 2208 | 315.000 | 315.000 |
| Corn starch | 367.400 | 349.200 |
| Carbomer 941 | 7.000 | 28.000 |
| Colloidal Silicon dioxide | 5.600 | 2.800 |
| Magnesium stearate | 3.500 | 3.500 |
| Total Mass | 700.000 | 700.000 |

### Example 8

The following Examples 6 to 14 show pramipexole tablet formulations which correspond to formulation b) providing a faster release characteristic for pH<4.5.

TABLE 8

| Constituents | mg/tablet |
|---|---|
| Pramipexole-dihydrochloride monohydrate, peg-milled | 0.750 |
| Hypromellose 2208 (Methocel K 15 M) | 175.000 |
| Carboxymethylcellulose sodium | 87.500 |
| Lactose monohydrate (200 mesh) | 52.500 |
| Microcrystalline cellulose (grade PH 101) | 31.100 |
| Colloidal silicon dioxide | 1.400 |
| Magnesium stearate | 1.750 |
| Total weight matrix tablet | 350.000 |

### Example 9

TABLE 9

| Constituents | mg/tablet |
|---|---|
| Pramipexole-dihydrochloride monohydrate, peg-milled | 0.750 |
| Hypromellose 2208 (Methocel K 15 M) | 175.000 |
| Carboxymethylcellulose sodium | 87.500 |
| Lactose monohydrate (200 mesh) | 52.500 |
| Microcrystalline cellulose (grade PH 101) | 27.600 |
| Carbomer 941 (Carbopol ® 71 G) | 3.500 |
| Colloidal silicon dioxide | 1.400 |
| Magnesium stearate | 1.750 |
| Total weight matrix tablet | 350.000 |

### Example 10

TABLE 10

| Constituents | mg/tablet |
|---|---|
| Pramipexole-dihydrochloride monohydrate, peg-milled | 0.750 |
| Hypromellose 2208 (Methocel K 15 M) | 175.000 |
| Carboxymethylcellulose sodium | 87.500 |
| Lactose monohydrate (200 mesh) | 45.500 |
| Microcrystalline cellulose (grade PH 101) | 24.100 |
| Carbomer 941 (Carbopol ® 71 G) | 14.000 |
| Colloidal silicon dioxide | 1.400 |
| Magnesium stearate | 1.750 |
| Total weight matrix tablet | 350.000 |

### Example 11

TABLE 11

| Constituents | mg/tablet |
|---|---|
| Pramipexole-dihydrochloride monohydrate, peg-milled | 0.750 |
| Carbomer 941 (Carbopol ® 71 G) | 87.500 |
| Lactose monohydrate (200 mesh) | 225.400 |
| Microcrystalline cellulose (grade PH 101) | 33.200 |
| Colloidal silicon dioxide | 1.400 |
| Magnesium stearate | 1.750 |
| Total weight matrix tablet | 350.000 |

### Example 12

TABLE 12

| Constituents | mg/tablet |
|---|---|
| Pramipexole-dihydrochloride monohydrate, peg-milled | 0.750 |
| Carbomer 941 (Carbopol ® 71 G) | 70.000 |
| Lactose monohydrate (200 mesh) | 242.900 |
| Microcrystalline cellulose (grade PH 101) | 33.200 |
| Colloidal silicon dioxide | 1.400 |
| Magnesium stearate | 1.750 |
| Total weight matrix tablet | 350.000 |

### Example 13

TABLE 13

| Constituents | mg/tablet |
|---|---|
| Pramipexole-dihydrochloride monohydrate, peg-milled | 0.750 |
| Carbomer 941 (Carbopol ® 71 G) | 70.000 |
| Lactose monohydrate (200 mesh) | 140.000 |
| Calcium Phosphate, dibasic dihydrate | 136.100 |
| Colloidal silicon dioxide | 1.400 |
| Magnesium stearate | 1.750 |
| Total weight matrix tablet | 350.000 |

### Example 14

The following Example shows a pramipexole tablet formulation which corresponds to formulation a) providing a release characteristic independent in the pH range of 1 to 7.5.

TABLE 14

| Constituents | mg/tablet |
|---|---|
| Pramipexole-dihydrochloride monohydrate, peg-milled | 0.750 |
| Hypromellose 2208 (Methocel K 100 M) | 157.500 |
| Corn starch | 187.900 |
| Colloidal silicon dioxide | 2.100 |
| Magnesium stearate | 1.750 |
| Total weight matrix tablet | 350.000 |

1. An extended release formulation comprising pramipexole or a pharmaceutically acceptable salt thereof having a reduced side effect profile when administered to a patient in terms of at least one condition selected from sleepiness and/or hallucinations and/or dizziness and/or headache and/or dyskinesia and/or obstipation and/or periphere oedema and/or nausea when compared to an immediate release formulation comprising pramipexole or a pharmaceutically acceptable salt thereof when the immediate release formulation is administered to the patient as often as needed to provide the same average blood plasma concentration of pramipexole in said patient as is achieved during the release period of the extended release formulation when the extended release formulation is administered once in the same period.

2. A method for treating a disease in a patient that is responsive to dopaminergic treatment, comprising administering to a patient having said disease an extended release formulation comprising pramipexole or pramipexoledichloride monohydrate or another pharmaceutically acceptable salt of pramipexole and said formulation having an at least partially pH-dependant release profile.

3. A method according to claim 2, wherein the pH-dependent release profile shows a faster release characteristic in the range of pH<4.5 and a slower and pH-independent release characteristic in the range from pH 4.5 to 7.5.

4. A method according to claim 2 wherein the formulation shows a reduced side effect profile when administered to a patient in terms of at least one condition selected from sleepiness and/or hallucinations and/or dizziness and/or headache and/or dyskinesia and/or obstipation and/or periphere oedema and/or nausea in comparison to an immediate release formulation comprising pramipexole or a pharmaceutically acceptable salt thereof when the immediate release formulation is administered to the patient as often as needed to provide the same average blood plasma concentration of pramipexole in said patient as is achieved during the release period of the extended release formulation when the extended release formulation is administered once in the same period.

5. A method according to claim 2 wherein the disease treated is idiopathic Parkinson's disease.

6. A method according to claim 2 wherein the disease treated is idiopathic Parkinson's disease with non-motor symptoms.

7. A method according to claim 2 wherein the disease treated is idiopathic Parkinson's disease and the treatment is in addition to another anti-Parkinson baseline treatment.

8. A method according to claim 2 wherein the patient has depressive symptoms.

9. A method according to claim 2 wherein the disease treated is idiopathic Parkinson's disease with motor symptoms.

10. A method according to claim 2 wherein the disease treated is idiopathic RLS.

11. A method according to claim 2 wherein the disease treated is Fibromyalgia.

12. A method according to claim 2 wherein the disease treated is Bipolar Disorder.

13. A formulation according to claim 1 wherein the extended release formulation comprises pramipexole or a pharmaceutically acceptable salt thereof in a matrix comprising at least one water swelling polymer other than pregelatinized starch.

14. A formulation according to claim 13, wherein the matrix comprises at least two water swelling polymers other than pregelatinized starch, and wherein at least one of the at least two polymers is an anionic polymer.

15. A formulation according to claim 14, wherein the anionic polymer is selected from the group consisting of optionally crosslinked acrylic acid polymers, methacrylic acid polymers, alginates and carboxymethylcellulose.

16. A formulation according to claim 15, wherein the anionic polymer is an optionally crosslinked acrylic acid polymer, and wherein the content of the optionally crosslinked acrylic acid polymer in the matrix is from about 0.25 wt.-% to about 25 wt.-%.

17. A formulation according to claim 13, comprising a substantially neutral polymer other than pregelatinized starch.

18. A formulation according to claim 17, wherein the substantially neutral polymer is selected from hydroxypropyl cellulose and hydroxypropylmethyl cellulose.

19. A formulation according to claim 18, wherein the substantially neutral polymer is hydroxypropyl methylcellulose, and wherein the content of hydroxypropyl methylcellulose in the matrix is from about 10 wt.-% to about 75 wt.-%.

20. A formulation according to claim 13, wherein the matrix comprises about:

| | | |
|---|---|---|
| (a) pramipexole or a salt thereof | 0.05 to 5 | wt.-%; |
| (b) anionic water swelling polymer(s) | 0.25 to 25 | wt.-%; |
| (c) neutral water swelling polymer(s) | 10 to 75 | wt.-%; and |
| (d) further excipients | ad 100 | wt.-%. |

21. A formulation according to claim 13, characterised in that the formulation is a tablet formulation comprising pramipexole or a pharmaceutically acceptable salt thereof in a matrix comprising
(a) at least one water swelling polymer other than pregelatinized starch and optionally excipients, the resulting tablet providing a pH-independent in vitro release characteristic in the range from pH 1 to 7.5, or
(b) at least one water swelling anionic polymer and optionally excipients, the resulting tablet providing a pH-dependent release characteristic with a faster release characteristic in the range of pH<4.5, and a slower and pH-independent release characteristic in the range from pH 4.5 to 7.5.

22. A method according to claim 2, characterised in that the extended release formulation is administered once daily.

23. A formulation according to claim 1, characterised in that the immediate release formulation is a tablet which comprises as inactive ingredients mannitol, corn starch, colloidal silicon dioxide, povidone, and magnesium stearate and as active ingredient pramipexole dihydrochloride monohydrate in an amount of either 0.125 mg or 0.25 mg or 0.5 mg or 1.0 mg or 1.5 mg or more.

24. A formulation according to claim 1, characterised in that the extended release formulation is in the form of a tablet.

25. A formulation according to claim 1, characterised in that the extended release formulation is in the form of a tablet having a non-functional coating.

26. A method for treating a disease in a patient that is responsive to dopaminergic treatment, comprising administering to a patient having said disease an extended release formulation comprising pramipexole or pramipexoledichloride monohydrate or another pharmaceutically acceptable salt of pramipexole and said formulation having an at least partially pH-independent in vitro release profile.

27. A method according to claim 26, characterised in that the release profile is pH independent over a pH range of pH 1 to 7.5.

28. A method according to claim 26, characterised in that the formulation comprises at least one water swelling polymer.

29. A method according to claim 28, characterised in that the water swelling polymer is a substantially neutral polymer.

30. A method according to claim 28, characterised in that the water swelling polymer is selected from the group consisting of alkylcelluloses; hydroxyalkylcelluloses,; hydroxyalkyl alkylcelluloses,; carboxyalkylcellulose esters; other natural, semi-synthetic, or synthetic di-, oligo- and polysaccharides; methacrylate copolymers; polyvinylalcohol; polyvinylpyrrolidone, copolymers of polyvinylpyrrolidone with vinyl acetate; combinations of polyvinylalcohol and polyvinylpyrrolidone; and polyalkylene oxides.

31. A method according to claim 28, characterised in that the formulation further comprises at least one additive selected from the group consisting of diluents or fillers, glidants, binding agents, granulating agents, anti-caking agents, lubricants, flavors, dyes,preservatives, and coating agents, provided that it does not comprise an ionic or anionic water swelling polymer in an amount that substantially impacts the pH release profile.

32. A method according to claim 26, wherein the formulation shows a reduced side effect profile when administered to a patient in terms of at least one condition selected from sleepiness and/or hallucinations and/or dizziness and/or headache and/or dyskinesia and/or obstipation and/or periphere oedema and/or nausea in comparison to an immediate release formulation comprising pramipexole or a pharmaceutically acceptable salt thereof when the immediate release formulation is administered to the patient as often as needed to provide the same average blood plasma concentration of pramipexole in said patient as is achieved during the release period of the extended release formulation when the extended release formulation is administered once in the same period.

33. A method according to claim 26 wherein the disease treated is idiopathic Parkinson's disease.

34. A method according to claim 26 wherein the disease treated is idiopathic Parkinson's disease with non-motor symptoms.

35. A method according to claim 26 wherein the disease treated is idiopathic Parkinson's disease and the treatment is in addition to another anti-Parkinson baseline treatment.

36. A method according to claim 26 wherein the patient has depressive symptoms.

37. A method according to claim 26 wherein the disease treated is idiopathic Parkinson's disease with motor symptoms.

38. A method according to claim 26 wherein the disease treated is idiopathic RLS.

39. A method according to claim 26 wherein the disease treated is Fibromyalgia.

40. A method according to claim 26 wherein the disease treated is Bipolar Disorder.
